(19) United States
(12) Patent Application Publication
(10) Pub. No.: US 2022/0002232 A1
(43) Pub. Date: Jan. 6, 2022

(54) PROCESS FOR THE PREPARATION OF 4,5-DIHYDROXY-2-(4-METHYLBENZYL)ISOPHTHALONITRILE

(71) Applicant: ORION CORPORATION, Espoo (FI)

(72) Inventors: Esa KUMPULAINEN, Helsinki (FI); IIpo LAITINEN, Espoo (FI); Jarmo PYSTYNEN, Espoo (FI)

(21) Appl. No.: 17/291,878

(22) PCT Filed: Nov. 8, 2019
(86) PCT No.: PCT/FI2019/050796

§ 371 Date: May 6, 2021

(30) Foreign Application Priority Data
Nov. 9, 2018 (FI) 20185953

(51) Int. Cl. C07C 253/00

(52) U.S. Cl. CPC C07C 253/00

(57) ABSTRACT

The present disclosure relates to a process for the preparation of 4,5-dihydroxy-2-(4-methylbenzyl)isophthalonitrile, to use of a compound which is 2-methoxy-5-(4-methylbenzyl)phenol, (3-hydroxy-4-methoxyphenyl)(p-tolyl)methanone, 2-methoxy-5-(4-methylbenzoyl)phenyl 2-chloroacetate, 4-methylbenzoyl chloride, 2-methoxyphenyl 2-chloroacetate or 2-methoxyphenol in the preparation of 4,5-dihydroxy-2-(4-methylbenzyl)isophthalonitrile and to a compound which is 4-hydroxy-5-methoxy-2-(4-methylbenzyl)isophthalaldehyde dioxime or 4-hydroxy-5-methoxy-2-(4-methylbenzyl)isophthalaldehyde and use thereof in the preparation of 4,5-dihydroxy-2-(4-methylbenzyl)isophthalonitrile. 4,5-Dihydroxy-2-(4-methylbenzyl)isophthalonitrile is a COMT inhibitor.

### FIELD OF THE INVENTION

The present disclosure relates to a process for the preparation of 4,5-dihydroxy-2-(4-methylbenzyl)isophthalonitrile, to use of a compound which is 2-methoxy-5-(4-methylbenzyl)phenol, (3-hydroxy-4-methoxyphenyl)(p-tolyl)methanone, 2-methoxy-5-(4-methylbenzoyl)phenyl 2-chloroacetate, 4-methylbenzoyl chloride, 2-methoxyphenyl 2-chloroacetate or 2-methoxyphenol in the preparation of 4,5-dihydroxy-2-(4-methylbenzyl)isophthalonitrile and to a compound which is 4-hydroxy-5-methoxy-2-(4-methylbenzyl)isophthalaldehyde dioxime or 4-hydroxy-5-methoxy-2-(4-methylbenzyl)isophthalaldehyde and use thereof in the preparation of 4,5-dihydroxy-2-(4-methylbenzyl)isophthalonitrile. 4,5-Dihydroxy-2-(4-methylbenzyl)isophthalonitrile is a catechol O-methyltransferase (COMT) inhibitor.

### BACKGROUND OF THE INVENTION

The compound 4,5-dihydroxy-2-(4-methylbenzyl)isophthalonitrile of formula (1A) has been disclosed in WO 2013/175053.

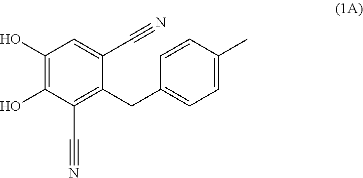 [SMILES: Cc1ccc(Cc2c(C#N)cc(O)c(O)c2C#N)cc1]

4,5-Dihydroxy-2-(4-methylbenzyl)isophthalonitrile is a COMT inhibitor. COMT inhibitors have been shown to be effective in clinical use for the treatment of Parkinson's disease as an adjunct to levodopa therapy. COMT inhibitors have also been indicated to be useful in the treatment of, for example, hypertension, heart failure and depression (U.S. Pat. No. 5,446,194) as well as inhibitors for the prevention of diabetic vascular dysfunctions (WO 98/27973). COMT inhibitors have also been disclosed as being useful for treating or controlling pain (WO 01/68083) as well as for treating restless legs syndrome (RLS), which is also known as Ekbom's syndrome (WO 2006/051154).

The process depicted in Scheme 1 for the preparation of 4,5-dihydroxy-2-(4-methylbenzyl)isophthalonitrile has been disclosed in WO 2013/175053.

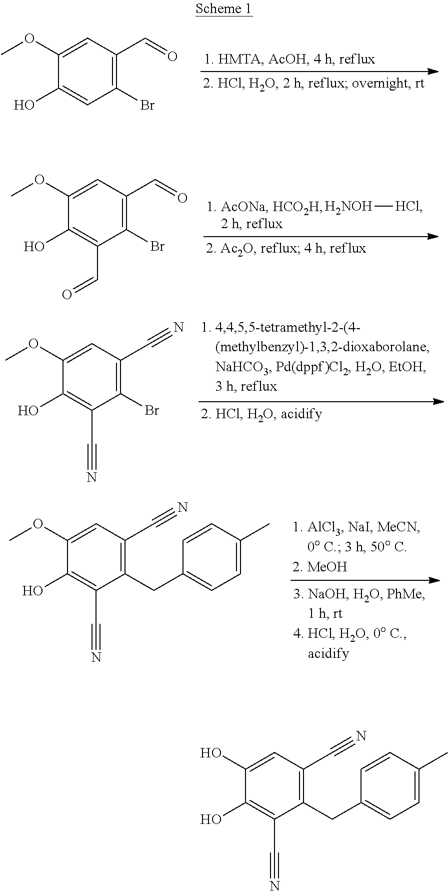

In Scheme 1, HMTA, AcOH, rt, AcONa, Ac₂O, Pd(dppf)Cl₂, EtOH, MeCN, MeOH and PhMe are hexamethylenetetramine, acetic acid, room temperature, sodium acetate, acetic anhydride, (1,1′-bis(diphenylphosphino)ferrocene)palladium dichloride, ethanol, acetonitrile, methanol and toluene, respectively.

The process depicted in Scheme 1 is associated with several drawbacks. The process involves use of a homogeneous palladium catalyst. This results in residual palladium and the catalyst is difficult to recycle. The commercial availability of the starting material, i.e. 2-bromo-4-hydroxy-5-methoxybenzaldehyde, in large quantities is limited. 2-Bromo-4-hydroxy-5-methoxybenzaldehyde can be prepared by bromination of 4-hydroxy-3-methoxybenzaldehyde with bromine. However, use of bromine is undesirable in large-scale production.

### SUMMARY OF THE INVENTION

The present disclosure provides a process for the preparation of 4,5-dihydroxy-2-(4-methylbenzyl)isophthalonitrile, or a pharmaceutically acceptable salt thereof, by converting 4-hydroxy-5-methoxy-2-(4-methylbenzyl)isophthalaldehyde dioxime to 4,5-dihydroxy-2-(4-methylbenzyl)isophthalonitrile and optionally converting 4,5-dihydroxy-2-(4-methylbenzyl)isophthalonitrile to a pharmaceutically acceptable salt thereof.

The present disclosure also provides use of a compound which is 2-methoxy-5-(4-methylbenzyl)phenol, (3-hydroxy-4-methoxyphenyl)(p-tolyl)methanone, 2-methoxy-5-(4-methylbenzoyl)phenyl 2-chloroacetate, 4-methylbenzoyl chloride, 2-methoxyphenyl 2-chloroacetate or 2-methoxyphenol in the preparation of 4,5-dihydroxy-2-(4-methylbenzyl)isophthalonitrile.

The present disclosure also provides a compound which is 4-hydroxy-5-methoxy-2-(4-methylbenzyl)isophthalaldehyde dioxime or 4-hydroxy-5-methoxy-2-(4-methylbenzyl)isophthalaldehyde and use thereof in the preparation of 4,5-dihydroxy-2-(4-methylbenzyl)isophthalonitrile.

The process for the preparation of 4,5-dihydroxy-2-(4-methylbenzyl)isophthalonitrile provided by the present disclosure does not involve use of a homogeneous palladium catalyst. Also, the starting material for the process is easily available in large quantities and the process does not involve use of bromine.

### DETAILED DESCRIPTION OF THE INVENTION

The present disclosure relates to a process for the preparation of 4,5-dihydroxy-2-(4-methylbenzyl)isophthalonitrile of formula (1A)

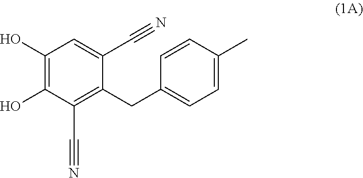 [SMILES: Cc1ccc(Cc2c(C#N)cc(O)c(O)c2C#N)cc1]

or a pharmaceutically acceptable salt thereof by converting 4-hydroxy-5-methoxy-2-(4-methylbenzyl)isophthalaldehyde dioxime of formula (VI′)

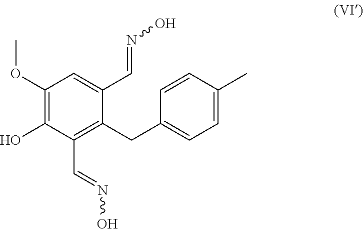 [SMILES: COc1cc(C=NO)c(Cc2ccc(C)cc2)c(C=NO)c1O]

to the compound of formula (1A)

and optionally converting the compound of formula (1A) to a pharmaceutically acceptable salt thereof.

In one embodiment, the present disclosure relates to a process, wherein the conversion of the compound of formula (VI′) to the compound of formula (1A) is carried out by converting the compound of formula (VI′) to 4-hydroxy-5-methoxy-2-(4-methylbenzyl)isophthalonitrile of formula (VI)

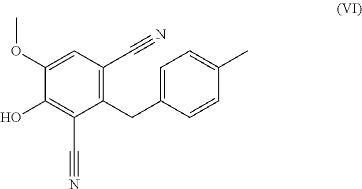 [SMILES: COc1cc(C#N)c(Cc2ccc(C)cc2)c(C#N)c1O]

and subsequently demethylating the compound of formula (VI) to obtain the compound of formula (1A).

In one embodiment, the present disclosure relates to a process, wherein the conversion of the compound of formula (VI′) to the compound of formula (VI) is carried out by reacting the compound of formula (VI′) with acetic anhydride.

In one embodiment, the present disclosure relates to a process, wherein the reaction of the compound of formula (VI′) with acetic anhydride is carried out in the presence of a weak base, e.g. sodium formate or sodium acetate, such as sodium formate.

In one embodiment, the present disclosure relates to a process, wherein the reaction of the compound of formula (VI′) with acetic anhydride is carried out in toluene, o-xylene, m-xylene, p-xylene or a mixture thereof, e.g. toluene.

In one embodiment, the present disclosure relates to a process, wherein the demethylation of the compound of formula (VI) to obtain the compound of formula (1A) is carried out by reacting the compound of formula (VI) with AlCl₃ in the presence of NaI.

In one embodiment, the present disclosure relates to a process, wherein the reaction of the compound of formula (VI) with AlCl₃ in the presence of NaI is carried out in acetonitrile.

In one embodiment, the present disclosure relates to a process, wherein the compound of formula (VI′) is prepared by converting 4-hydroxy-5-methoxy-2-(4-methylbenzyl)isophthalaldehyde of formula (V)

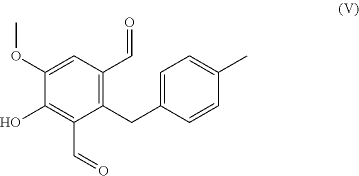 [SMILES: COc1cc(C=O)c(Cc2ccc(C)cc2)c(C=O)c1O]

to the compound of formula (VI′).

In one embodiment, the present disclosure relates to a process, wherein the conversion of the compound of formula (V) to the compound of formula (VI′) is carried out by reacting the compound of formula (V) with hydroxylamine water solution.

In one embodiment, the present disclosure relates to a process, wherein the reaction of the compound of formula (V) with hydroxylamine water solution is carried out in the presence of an acid, e.g. acetic acid.

In one embodiment, the present disclosure relates to a process, wherein the reaction of the compound of formula (V) with hydroxylamine water solution is carried out in methanol, acetonitrile, ethanol, propan-2-ol or a mixture thereof, e.g. acetonitrile.

In one embodiment, the present disclosure relates to a process, wherein the compound of formula (V) is prepared by converting (3-hydroxy-4-methoxyphenyl)(p-tolyl)methanone of formula (III)

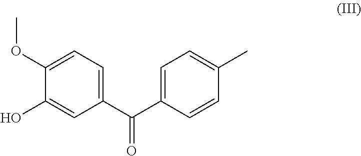 [SMILES: COc1ccc(C(=O)c2ccc(C)cc2)cc1O]

to the compound of formula (V).

In one embodiment, the present disclosure relates to a process, wherein the conversion of the compound of formula (III) to the compound of formula (V) is carried out by reducing the compound of formula (III) to obtain 2-methoxy-5-(4-methylbenzyl)phenol of formula (IV)

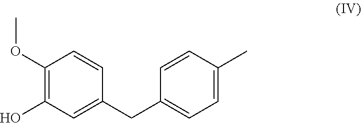 [SMILES: COc1ccc(Cc2ccc(C)cc2)cc1O]

and subsequently formylating the compound of formula (IV) to obtain the compound of formula (V).

In one embodiment, the present disclosure relates to a process, wherein the reduction of the compound of formula (III) to obtain the compound of formula (IV) is carried out by hydrogenating the compound of formula (III) in the presence of palladium on carbon.

Heterogeneous palladium on carbon is easily separated by filtration and can be recycled.

In one embodiment, the present disclosure relates to a process, wherein the hydrogenation of the compound of formula (III) is carried out in acetic acid.

In one embodiment, the present disclosure relates to a process, wherein the formylation of the compound of formula (IV) to obtain the compound of formula (V) is carried out by reacting the compound of formula (IV) with hexamethylenetetramine.

In one embodiment, the present disclosure relates to a process, wherein the reaction of the compound of formula (IV) with hexamethylenetetramine is carried out in a mixture of acetic acid and water.

In one embodiment, the present disclosure relates to a process, wherein the compound of formula (III) is prepared by converting 2-methoxyphenol of formula (Ia)

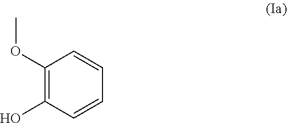

to the compound of formula (III).

In one embodiment, the present disclosure relates to a process, wherein the conversion of the compound of formula (Ia) to the compound of formula (III) is carried out by reacting the compound of formula (Ia) with 2-chloroacetyl chloride of formula (Ib)

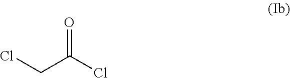

to obtain 2-methoxyphenyl 2-chloroacetate of formula (I)

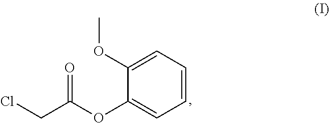

subsequently reacting the compound of formula (I) with 4-methylbenzoyl chloride of formula (IIa)

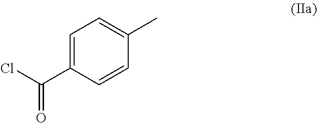

to obtain 2-methoxy-5-(4-methylbenzoyl)phenyl 2-chloroacetate of formula (II)

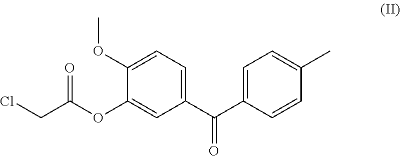 [SMILES: COc1ccc(C(=O)c2ccc(C)cc2)cc1OC(=O)CCl]

and subsequently converting the compound of formula (II) to the compound of formula (III). 2-Methoxyphenol and 4-methylbenzoyl chloride are easily available in large quantities.

In one embodiment, the present disclosure relates to a process, wherein the reaction of the compound of formula (Ia) with the compound of formula (Ib) is carried out in the presence of NaOH, KOH, Na₂CO₃ or K₂CO₃, e.g. NaOH.

In one embodiment, the present disclosure relates to a process, wherein the reaction of the compound of formula (Ia) with the compound of formula (Ib) is carried out in the presence of NaOH, wherein the amount of NaOH used per amount of the compound of formula (Ta) is 1-2 molar equivalents.

In one embodiment, the present disclosure relates to a process, wherein the reaction of the compound of formula (I) with the compound of formula (IIa) is carried out in the presence of a Lewis acid, e.g. AlCl₃.

In one embodiment, the present disclosure relates to a process, wherein the reaction of the compound of formula (Ia) with the compound of formula (Ib) and the reaction of the compound of formula (I) with the compound of formula (IIa) are carried out in chloro(C₁₋₂)alkane, e.g. dichloromethane, trichloromethane, 1,2-dichloroethane or a mixture thereof, such as dichloromethane.

In one embodiment, the present disclosure relates to a process, wherein the conversion of the compound of formula (II) to the compound of formula (III) is carried out by reacting the compound of formula (II) with methanol in the presence of an acid, e.g. HCl.

In one embodiment, the present disclosure relates to a process, wherein the compound of formula (1A) prepared according to any of the embodiments above is crystallized from a mixture of ethanol and water, e.g. from a mixture, wherein the amount of water in the mixture of ethanol and water is 50-90 volume-%.

In one embodiment, the present disclosure relates to use of a compound which is 2-methoxy-5-(4-methylbenzyl)phenol of formula (IV)

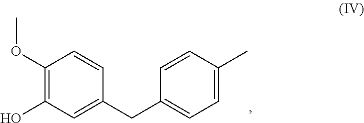 [SMILES: COc1ccc(Cc2ccc(C)cc2)cc1O]

(3-hydroxy-4-methoxyphenyl)(p-tolyl)methanone of formula (III)

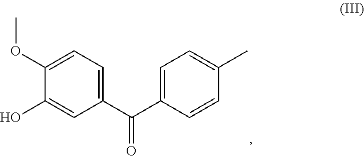 [SMILES: COc1ccc(C(=O)c2ccc(C)cc2)cc1O]

2-methoxy-5-(4-methylbenzoyl)phenyl 2-chloroacetate of formula (II)

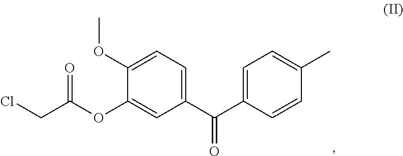 [SMILES: COc1ccc(C(=O)c2ccc(C)cc2)cc1OC(=O)CCl]

4-methylbenzoyl chloride of formula (IIa)

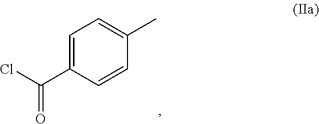

2-methoxyphenyl 2-chloroacetate of formula (I)

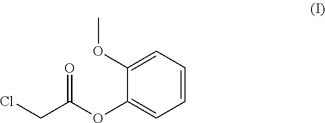

or 2-methoxyphenol of formula (Ia)

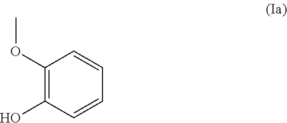

in the preparation of 4,5-dihydroxy-2-(4-methylbenzyl)isophthalonitrile of formula (1A)

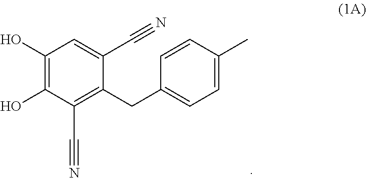 [SMILES: Cc1ccc(Cc2c(C#N)cc(O)c(O)c2C#N)cc1]

In one embodiment, the present disclosure relates to use of a compound which is 2-methoxy-5-(4-methylbenzyl)phenol in the preparation of 4,5-dihydroxy-2-(4-methylbenzyl)isophthalonitrile.

In one embodiment, the present disclosure relates to use of a compound which is (3-hydroxy-4-methoxyphenyl)(p-tolyl)methanone in the preparation of 4,5-dihydroxy-2-(4-methylbenzyl)isophthalonitrile.

In one embodiment, the present disclosure relates to use of a compound which is 2-methoxy-5-(4-methylbenzoyl)phenyl 2-chloroacetate in the preparation of 4,5-dihydroxy-2-(4-methylbenzyl)isophthalonitrile.

In one embodiment, the present disclosure relates to use of a compound which is 4-methylbenzoyl chloride in the preparation of 4,5-dihydroxy-2-(4-methylbenzyl)isophthalonitrile.

In one embodiment, the present disclosure relates to use of a compound which is 2-methoxyphenyl 2-chloroacetate in the preparation of 4,5-dihydroxy-2-(4-methylbenzyl)isophthalonitrile.

In one embodiment, the present disclosure relates to use of a compound which is 2-methoxyphenol in the preparation of 4,5-dihydroxy-2-(4-methylbenzyl)isophthalonitrile.

In one embodiment, the present disclosure relates to a compound which is 4-hydroxy-5-methoxy-2-(4-methylbenzyl)isophthalaldehyde dioxime of formula (VI′)

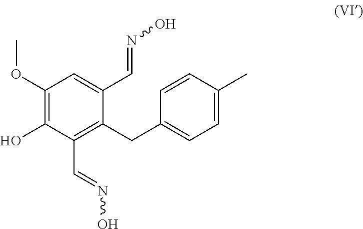 [SMILES: COc1cc(C=NO)c(Cc2ccc(C)cc2)c(C=NO)c1O]

or 4-hydroxy-5-methoxy-2-(4-methylbenzyl)isophthalaldehyde of formula (V)

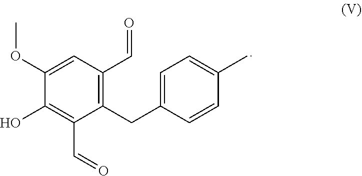 [SMILES: COc1cc(C=O)c(Cc2ccc(C)cc2)c(C=O)c1O]

In one embodiment, the present disclosure relates to a compound, wherein the compound is 4-hydroxy-5-methoxy-2-(4-methylbenzyl)isophthalaldehyde dioxime.

In one embodiment, the present disclosure relates to a compound, wherein the compound is 4-hydroxy-5-methoxy-2-(4-methylbenzyl)isophthalaldehyde.

In one embodiment, the present disclosure relates to use of a compound which is 4-hydroxy-5-methoxy-2-(4-methylbenzyl)isophthalaldehyde dioxime or 4-hydroxy-5-methoxy-2-(4-methylbenzyl)isophthalaldehyde in the preparation of 4,5-dihydroxy-2-(4-methylbenzyl)isophthalonitrile of formula (1A)

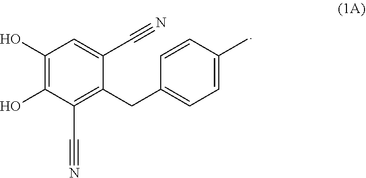 [SMILES: Cc1ccc(Cc2c(C#N)cc(O)c(O)c2C#N)cc1]

The terms employed herein have the meanings indicated below. The term “at least one chlorine” employed in the meanings below refers to one or several chlorine(s).

The term “weak base”, as employed herein, refers to a proton acceptor that is only partially dissociated in an aqueous solution. Representative examples of weak bases include, but are not limited to, sodium formate, sodium acetate and triethylamine The term “chloro(C₁₋₂)alkane”, as employed herein, refers to at least one chlorine appended to methane or ethane. When there are several chlorines, the chlorines can be attached to different carbon atoms or several chlorines can be attached to the same carbon atom. Representative examples of chloro(C₁₋₂)alkane include, but are not limited to, dichloromethane, trichloromethane and 1,2-dichloroethane.

The term “Lewis acid”, as employed herein, refers to an electron-pair acceptor. Representative examples of Lewis acids include, but are not limited to, AlCl₃ and BBr₃.

The process for the preparation of 4,5-dihydroxy-2-(4-methylbenzyl)isophthalonitrile provided by the present disclosure does not involve use of a homogeneous palladium catalyst. Also, the process does not involve use of bromine.

The present disclosure is explained in more detail by the following examples. The examples are meant for illustrating purposes only and do not limit the scope of the invention defined in the claims.

The abbreviations have the meanings indicated below.

DMSO dimethyl sulfoxide

HPLC high-performance liquid chromatography

rt room temperature

NMR spectrum multiplicities have the meanings indicated below.

br s broad singlet

d doublet

s singlet

### Example 1: Preparation of 2-methoxyphenyl 2-chloroacetate

2-Methoxyphenol (20 ml), dichloromethane (60 ml) and water (28 ml) were charged. 50% NaOH (8.0 ml) was added slowly at 0-10° C. 2-Chloroacetyl chloride (10.0 ml) in dichloromethane (20 ml) was added slowly at 0-10° C. 50% NaOH (7.8 ml) was added at 0-10° C. 2-Chloroacetyl chloride (9.0 ml) in dichloromethane (10 ml) was added slowly at 0-10° C. The mixture was stirred about 1 h at 0-10° C. 30% HCl (6 ml) and water (60 ml) were added at 0-10° C. The aqueous phase was separated off. The organic phase was washed with water (60 ml). 60 ml of dichloromethane was distilled off Dichloromethane (100 ml) was added. 60 ml of dichloromethane was distilled off. The solution was used straight in the next step.

### Example 2: Preparation of 2-methoxy-5-(4-methylbenzoyl)phenyl 2-chloroacetate

Dichloromethane (60 ml) and aluminium chloride (14.8 g) were charged. 4-Methylbenzoyl chloride (16 ml) was added slowly at 0-10° C. Half of the solution obtained in Example 1 was added slowly at rt. The mixture was stirred overnight. Water (70 ml) and 30% HCl (16 ml) were added slowly at 0-10° C. The aqueous phase was separated off. The solution was used straight in the next step.

### Example 3: Preparation of (3-hydroxy-4-methoxyphenyl)(p-tolyl)methanone

50 ml of dichloromethane was distilled off from the solution obtained in Example 2. Methanol (132 ml) and 30% HCl (4.0 ml) were added. About 48 ml was distilled off. The mixture was refluxed for 2 h and then cooled to 0-5° C. The compound was filtered, washed with methanol (30 ml) and dried under reduced pressure at 50-60° C. The yield was 85.5% and the HPLC purity 99.9%.

### Example 4: Preparation of 2-methoxy-5-(4-methylbenzyl)phenol

(3-Hydroxy-4-methoxyphenyl)(p-tolyl)methanone (200 g), acetic acid (600 ml), palladium 5% on carbon and 50% water paste (18.3 g) were charged. The mixture was flushed several times with nitrogen and then hydrogenated at 3.0 bar overpressure of hydrogen for 2 h at about 65° C. The catalyst was filtered off under nitrogen. The cake was washed with acetic acid (343 ml). The solution was used straight in the next step.

### Example 5: Preparation of 4-hydroxy-5-methoxy-2-(4-methylbenzyl)isophthalaldehyde

500 ml of the solution obtained in Example 4 was charged. Hexamethylenetetramine (73 g) and water (30 ml) were added. The mixture was stirred for 5 h at about 120° C. 93 ml of the solution and water (2.5 ml) were charged. 30% HCl (24 ml) was added slowly at rt. The mixture was stirred overnight. The compound was filtered, washed with acetic acid (7 ml) and water (14 ml) and dried under reduced pressure at 55° C. The yield was 38.9% and the HPLC purity 96.4%. ¹H-NMR (400 MHz, d₆-DMSO): δ 2.22 (s, 3H), 3.92 (s, 3H), 4.77 (s, 2H), 6.93 (d, 2H), 7.06 (d, 2H), 7.63 (s, 1H), 10.24 (s, 1H), 10.44 (s, 1H), 12.01 (s, 1H).

### Example 6: Preparation of 4-hydroxy-5-methoxy-2-(4-methylbenzyl)isophthalaldehyde dioxime

4-Hydroxy-5-methoxy-2-(4-methylbenzyl)isophthalaldehyde (10 g), acetic acid (1.0 ml) and acetonitrile (50 ml) were charged. 10% Hydroxylamine water solution (28 ml) was added slowly at about 60° C. The mixture was stirred for 2 h at about 60° C. Water (33 ml) was added slowly at about 60° C. The mixture was cooled gradually to 0-5° C. and stirred for 2 h at 0-5° C. The compound was filtered, washed with acetonitrile:water (1:1) mixture (20 ml) and dried under reduced pressure at about 65° C. The yield was 90.8% and the HPLC purity 99.5%. ¹H-NMR (400 MHz, d₆-DMSO): δ 2.23 (s, 3H), 3.82 (s, 3H), 4.23 (s, 2H), 6.87 (d, 2H), 7.06 (d, 2H), 7.36 (s, 1H), 8.26 (s, 1H), 8.42 (s, 1H), 10.97 (br s, 1H), 11.05 (s, 1H), 11.61 (br s, 1H).

### Example 7: Preparation of 4-hydroxy-5-methoxy-2-(4-methylbenzyl)isophthalonitrile

4-Hydroxy-5-methoxy-2-(4-methylbenzyl)isophthalaldehyde dioxime (10 g) and toluene (45 ml) were charged. Acetic anhydride (7.0 ml) was added slowly at about 100° C. The mixture was stirred for 2 h at about 100° C. and then cooled to rt. Sodium formate (1.2 g) was added. The mixture was heated to 110° C., stirred for 6 h and cooled to rt. Acetonitrile (16 ml) was added. Water (10 ml) and 30% HCl (5 ml) were added slowly at about 80° C. The aqueous phase was separated off. The organic phase was cooled slowly to 0-5° C. and seeded during cooling. Hexane (15 ml) was added slowly at 0-5° C. The mixture was stirred for about 3 h at 0-5° C. The compound was filtered, washed with cold toluene (20 ml) and dried under reduced pressure at 70-80° C. The yield was 90.4% and the HPLC purity 99.6%.

### Example 8: Preparation of 4,5-dihydroxy-2-(4-methylbenzyl)isophthalonitrile

Acetonitrile (56 ml), aluminium chloride (8 g) and sodium iodide (9.5 g) were charged. 4-hydroxy-5-methoxy-2-(4-methylbenzyl)isophthalonitrile (10 g) was added. The mixture was heated to 45° C., stirred for 4 h and cooled to 15° C. Water (60 ml) and 30% HCl (15 ml) were added slowly at 15° C. Sodium sulphite (2 g) was added and the mixture was stirred for 90 min at 22° C. The phases were allowed to settle and the aqueous phase was separated off Water (30 ml), sodium chloride (3 g), sodium sulphite (1 g) and 30% HCl (1.5 ml) were added. The mixture was stirred for 1 h at 22° C. and the phases were allowed to settle. The aqueous phase was separated off. Solvents were distilled off under atmospheric pressure until the volume of the residue was 20 ml. Ethanol (80 ml) was added and the distillation was continued until the volume of the residue was 30 ml. The residue was cooled to 70° C. and ethanol (16 ml) and water (65 ml) were added. The mixture was cooled to 0° C. in 9 h and stirred for at least 1 h at 0° C. The product was filtered, washed with water (15 ml) and dried under reduced pressure at 35-40° C. in an agitated dryer. The yield was 92.5% and the HPLC purity 99.5%.

1. A process for the preparation of 4,5-dihydroxy-2-(4 methylbenzyl)isophthalonitrile of formula (1A) 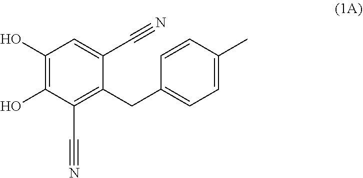 [SMILES: Cc1ccc(Cc2c(C#N)cc(O)c(O)c2C#N)cc1]
or a pharmaceutically acceptable salt thereof comprising:
converting 4-hydroxy-5-methoxy-2-(4-methylbenzyl)isophthalaldehyde dioxime of formula (VI′); 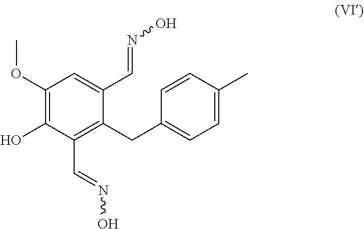 [SMILES: COc1cc(C=NO)c(Cc2ccc(C)cc2)c(C=NO)c1O]
to the compound of formula (1A);
and optionally converting the compound of formula (1A) to a pharmaceutically acceptable salt thereof.

2. The process according to claim 1, wherein the conversion of the compound of formula (VI′) to the compound of formula (1A) is carried out by converting the compound of formula (VI′) to 4-hydroxy-5-methoxy-2-(4-methylbenzyl)isophthalonitrile of formula (VI); 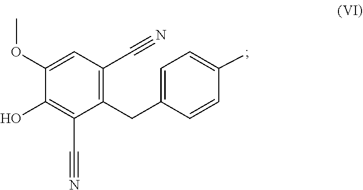
and subsequently demethylating the compound of formula (VI) to obtain the compound of formula (1A).

3. The process according to claim 2, wherein the conversion of the compound of formula (VI′) to the compound of formula (VI) is carried out by reacting the compound of formula (VI′) with acetic anhydride.

4. The process according to claim 3, wherein the reaction of the compound of formula (VI′) with acetic anhydride is carried out in the presence of a weak base.

5. The process according to claim 4, wherein the weak base is sodium formate or sodium acetate.

6. The process according to claim 5, wherein the weak base is sodium formate.

7. The process according to claim 3, wherein the reaction of the compound of formula (VI′) with acetic anhydride is carried out in toluene, o-xylene, m-xylene, p-xylene or a mixture thereof.

8. The process according to claim 7, wherein the reaction of the compound of formula (VI′) with acetic anhydride is carried out in toluene.

9. The process according to claim 2, wherein the demethylation of the compound of formula (VI) to obtain the compound of formula (1A) is carried out by reacting the compound of formula (VI) with AlCl₃ in the presence of NaI.

10. The process according to claim 9, wherein the reaction of the compound of formula (VI) with AlCl₃ in the presence of NaI is carried out in acetonitrile.

11. The process according to claim 1, wherein the compound of formula (VI′) is prepared by converting 4-hydroxy-5-methoxy-2-(4-methylbenzyl)isophthalaldehyde of formula (V) 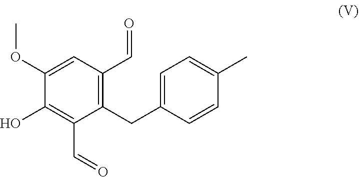 [SMILES: COc1cc(C=O)c(Cc2ccc(C)cc2)c(C=O)c1O]
to the compound of formula (VI′).

12. The process according to claim 11, wherein the conversion of the compound of formula (V) to the compound of formula (VI′) is carried out by reacting the compound of formula (V) with hydroxylamine water solution.

13. The process according to claim 12, wherein the reaction of the compound of formula (V) with hydroxylamine water solution is carried out in the presence of an acid.

14. The process according to claim 13, wherein the acid is acetic acid.

15. The process according to claim 12, wherein the reaction of the compound of formula (V) with hydroxylamine water solution is carried out in methanol, acetonitrile, ethanol, propan-2-ol or a mixture thereof.

16. The process according to claim 15, wherein the reaction of the compound of formula (V) with hydroxylamine water solution is carried out in acetonitrile.

17. The process according to claim 11, wherein the compound of formula (V) is prepared by converting (3-hydroxy-4-methoxyphenyl)(p-tolyl)methanone of formula (III); 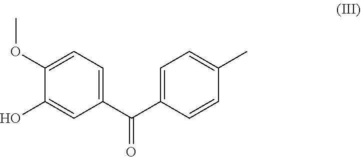 [SMILES: COc1ccc(C(=O)c2ccc(C)cc2)cc1O]
to the compound of formula (V).

18. The process according to claim 17, wherein the conversion of the compound of formula (III) to the compound of formula (V) is carried out by reducing the compound of formula (III) to obtain 2-methoxy-5-(4-methylbenzyl)phenol of formula (IV): 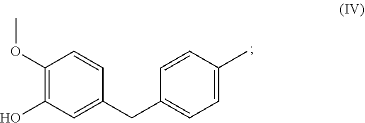 [SMILES: COc1ccc(Cc2ccc(C)cc2)cc1O]
and subsequently formylating the compound of formula (IV) to obtain the compound of formula (V).

19. The process according to claim 18, wherein the reduction of the compound of formula (III) to obtain the compound of formula (IV) is carried out by hydrogenating the compound of formula (III) in the presence of palladium on carbon.

20. The process according to claim 19, wherein the hydrogenation of the compound of formula (III) is carried out in acetic acid.

21. The process according to claim 18, wherein the formylation of the compound of formula (IV) to obtain the compound of formula (V) is carried out by reacting the compound of formula (IV) with hexamethylenetetramine.

22. The process according to claim 21, wherein the reaction of the compound of formula (IV) with hexamethylenetetramine is carried out in a mixture of acetic acid and water.

23. The process according to claim 17, wherein the compound of formula (III) is prepared by converting 2-methoxyphenol of formula (Ia); 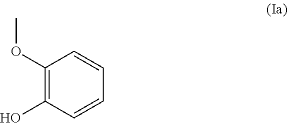
to the compound of formula (III).

24. The process according to claim 23, wherein the conversion of the compound of formula (Ia) to the compound of formula (III) is carried out by reacting the compound of formula (Ia) with 2-chloroacetyl chloride of formula (Ib): 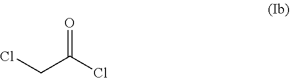
to obtain 2-methoxyphenyl 2-chloroacetate of formula (I); 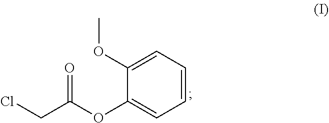
subsequently reacting the compound of formula (1) with 4-methylbenzoyl chloride of formula (IIa); 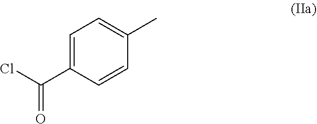
to obtain 2-methoxy-5-(4-methylbenzoyl)phenyl 2-chloroacetate of formula (II); 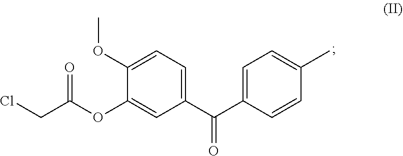 [SMILES: COc1ccc(C(=O)c2ccc(C)cc2)cc1OC(=O)CCl]
and subsequently converting the compound of formula (II) to the compound of formula (III).

25. The process according claim 24, wherein the reaction of the compound of formula (Ia) with the compound of formula (Ib) is carried out in the presence of NaOH, KOH, Na₂CO₃₁ or K₂CO₃.

26. The process according claim 25, wherein the reaction of the compound of formula (Ia) with the compound of formula (Ib) is carried out in the presence of NaOH.

27. The process according to claim 26, wherein the amount of NaOH used per amount of the compound of formula (Ia) is an amount ranging from 1 to 2 molar equivalents.

28. The process according to claim 24, wherein the reaction of the compound of formula (I) with the compound of formula (IIa) is carried out in the presence of a Lewis acid.

29. The process according to claim 28, wherein the Lewis acid is AlCl₃.

30. The process according to claim 28, wherein the reaction of the compound of formula (Ia) with the compound of formula (Ib) and the reaction of the compound of formula (1) with the compound of formula (IIa) are carried out in chloro(C₁₋₂)alkane.

31. The process according to claim 30, wherein the chloro(C₁₋₂)alkane is dichloromethane, trichloromethane, 1,2-dichloroethane, or a mixture thereof.

32. The process according to claim 31, wherein the chloro(C₁₋₂)alkane is dichloromethane.

33. The process according to claim 24, wherein the conversion of the compound of formula (II) to the compound of formula (III) is carried out by reacting the compound of formula (II) with methanol in the presence of an acid.

34. The process according to claim 33, wherein the acid is HCl.

35. The process according to claim 1, wherein the compound of formula (1A) is crystallized from a mixture of ethanol and water.

36. The process according to claim 35, wherein the amount of water in the mixture of ethanol and water is 50-90 volume-%.

37. (canceled)

38. A compound chosen from 4-hydroxy-5-methoxy-2-(4-methylbenzyl)isophthalaldehyde dioxime of formula (VI′) 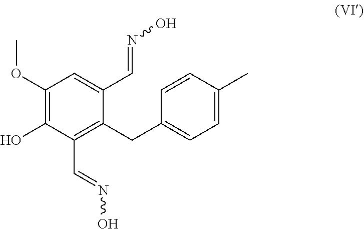 [SMILES: COc1cc(C=NO)c(Cc2ccc(C)cc2)c(C=NO)c1O]
and 4-hydroxy-5-methoxy-2-(4-methylbenzyl)isophthalaldehyde of formula (V)

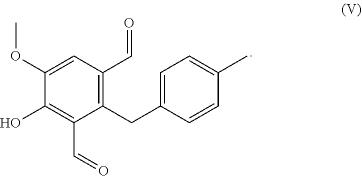 [SMILES: COc1cc(C=O)c(Cc2ccc(C)cc2)c(C=O)c1O]

39. The compound according to claim 38, wherein the compound is 4-hydroxy-5-methoxy-2-(4-methylbenzyl)isophthalaldehyde dioxime.

40. The compound according to claim 38, wherein the compound is 4-hydroxy-5-methoxy-2-(4-methylbenzyl)isophthalaldehyde.
